(19) United States
(12) Patent Application Publication
(10) Pub. No.: US 2009/0311760 A1
(43) Pub. Date: Dec. 17, 2009

(54) HOST CELLS AND USES THEREOF IN THE MICROBIAL PRODUCTION OF HYDROXYLATED AROMATICS

(75) Inventors: Jan Wery, Grossel (NL); Regina Gerda Maaike Westerhof, Deventer (NL); Karin Nijkamp, Rijssen (NL)

Correspondence Address: WEINGARTEN, SCHURGIN, GAGNEBIN & LEBOVICI LLP, TEN POST OFFICE SQUARE, BOSTON, MA, 02109, US

(73) Assignee: Schoemakerstraat 97, Delft (NL)

(21) Appl. No.: 12/301,105

(22) PCT Filed: May 18, 2007
(86) PCT No.: PCT/NL07/50230

§ 371 Date: Apr. 2, 2009

(30) Foreign Application Priority Data
May 17, 2006 (EP) 06076066.7

(51) Int. Cl. C12P 7/40; C12P 7/22; C12N 1/21

(52) U.S. Cl. 435/136; 435/156; 435/252.3

(57) ABSTRACT

The invention relates to the field of the microbial production of substituted aromatics. In particular, it relates to the production of hydroxylated aromatics from renewable carbon stocks, like sugars or glycerol, via the metabolic intermediate L-tyrosine. Provided is a microbial host cell capable of producing at least one hydroxylated aromatic from a renewable carbon source, wherein at least one enzyme of said host cell that is involved in the degradation of said at least one hydroxylated aromatic is disabled and wherein the de novo synthesis of L-phenylalanine (L-Phe) in said host cell is impeded. Also provided is a method for the microbial production of at least one hydroxylated aromatic from a renewable carbon source, comprising culturing a host cell in the presence of exogenous L-Phe and a renewable carbon source and allowing said host cell to produce said at least one hydroxylated aromatic.

The invention relates to the field of the microbial production of substituted aromatics. In particular, it relates to the production of hydroxylated aromatics from renewable carbon stocks, like sugars or glycerol, via the metabolic intermediate L-tyrosine.

There is a growing interest in developing biotechnological processes for the production of chemicals from renewable resources (Schmidt et al. 2001; Zaks, 2001; Maury et al., 2005). The main envisaged advantages of such “green” processes are reduction of the use of fossil fuels and less waste (e.g. CO₂) production (Anastas et al. 2002). Various laboratories are developing whole-cell bioprocesses for the production of substituted aromatics such as p-hydroxybenzoic acid (PHB) (Barker and Frost, 2001, WO2005/103273), phenol (Wierckx et al., 2005), cinnamic acid (CA) (Nijkamp et al., 2005, WO2005/103273), p-hydroxycinnamic acid (PHCA; also referred to as p-coumaric acid) and p-hydroxystyrene (PHS) (US2001/0053847; US2003/0079255; WO03/099233; US2004/0229326; WO2005/103273). Substituted aromatics are a very important class of chemicals in terms of their broad application. Examples of commercially important aromatics include CA, PHCA, PHB, PHS and p-hydroxystyreneoxide (PHSO).

PHCA is the precursor of various phenylpropanoids, such as lignins, flavonoids and coumarins in plants (Hanson and Havir, 1978; Hahlbrock and Scheel, 1989). It is a useful monomer for the production of Liquid Crystal Polymers (LCP). LCPs may be used in electronic connectors and telecommunication and aerospace applications. LCP resistance to sterilizing radiation has also enabled these materials to be used in medical devices as well as chemical, and food packaging applications. Furthermore, PHCA can be used in sunscreen products and cosmetics and as antioxidant in food stuff. An important pharmaceutical for high blood pressure and stroke prevention, known as coumarin or oxy-cinnamic acid, is a derivative of CA. PHCA is also a useful bio-monomer for biological and medical applications as degradable plastic, orthopaedic matrix, tissue engineering and drug delivery systems (Matsusaki et al., 2001; Kaneko et al., 2004; Matsusaki et al., 2005).

PHB is used as a monomer for synthesis of LCPs. It is also a food preservative and it is used as a stabilizer in cosmetic preparations. Also, PHB can serve as chemical intermediate for synthetic drugs, pharmaceuticals, dyes and plasticizers. Esters of PHB are known as parabens, which are used as antimicrobial preservatives in deodorants, antiperspirants and in a wide range of other consumer products. PHS has a utility in the production of, among others, resins, coatings and inks.

As the market for aromatics is huge and their chemical synthesis is oftentimes cumbersome, demanding much energy and/or expensive chemical activating and protection groups and/or large amounts of solvents, the bio-based production processes of these chemicals from renewable resources could provide a green and economically feasible alternative.

The microbial production of substituted aromatics is known in the art. For example, we previously disclosed the microbial production of aromatics using a Pseudomonas host cell (WO2005/103273). P. putida is a metabolically versatile bacterium that has considerable potential for biotechnological applications (Jimenez et al. 2002, Wackett, 2003). This specific P. putida strain is solvent-tolerant and able to actively extrude a variety of compounds by means of a solvent pump (Isken and De Bont, 1996; Kieboom et al., 1998), which could serve as a driver of biocatalytic conversions by exporting the product from the cell into the medium (WO2005/103273). Also for bioprocesses involving such products the use of solvent-tolerant P. putida strains renders advantages in terms of productivity and the application of multi-phase media for product recovery (Wery et al., 2000; Ramos-Gonzales et al., 2003, Rojas et al., 2004; Wierckx et al., 2005; Wery and De Bont, 2004)

The bio-based production of PHCA and PHS has been achieved by others in non-solvent-tolerant bacteria, such as Escherichia coli and/or Pseudomonas aeruginosa that were genetically modified to express the pal-gene encoding PAL from e.g. Rhodosporidium toruloides (DuPont: US2001/0053847 A1, U.S. Pat. No. 6,368,837B1, US2003/0079255 A1, WO03/099233 A2, US2004/0229326 A1). The enzyme PAL (EC 4.3.1.5) catalyzes the conversion of L-phenylalanine and L-tyrosine to CA and PHCA, respectively. The production of CA from glucose was previously achieved upon introduction of PAL activity in P. putida S12. It was shown that PHCA was also produced, albeit transiently and in minute quantities (Nijkamp et al., 2005).

Production of substituted aromatics based solely on the heterologous expression of the pal gene has a major disadvantage, which lies in the intrinsic quality of PAL to convert both intracellular L-phenylalanine and L-tyrosine to respectively CA and PHCA with similar efficiency. Typically, PAL-based microbiological production of hydroxylated aromatics via L-tyrosine, like PHCA and PHS and derivatives thereof, suffer from formation of the by-product CA from L-phenylalanine. Moreover, by virtue of their similar molecular structures and physico-chemical properties, the desired product(s) and the by-products(s), like CA and PHCA, are typically difficult to separate during downstream processing procedures.

Therefore, a key issue for efficient production of these hydroxylated aromatics using microbial host cells is, besides optimizing carbon flux towards these products, decreasing the formation of the by-product CA that arises from the action of PAL.

Several methods have been described in attempt to achieve this, see for example US2001/0053847 A1, U.S. Pat. No. 6,368,837B1, US2003/0079255 A1, WO03/099233 A2, US2004/0229326 A1. The known approaches involve 1. Heterologous expression of gene(s) encoding L-phenylalanine hydroxylase which converts L-phenylalanine into L-tyrosine. 2. Heterologous expression of gene(s) encoding CA-4-hydroxylase (p450-reductase), which converts CA into PHCA. 3. Modification of the pal gene such that it only uses L-tyrosine and no longer L-phenylalanine as a substrate (=L-tyrosine ammonia lyase, TAL).

These known approaches suffer from several disadvantages. For example, L-phenylalanine hydroxylase and CA-4-hydroxylase require energy (NAD(P)H) for their reaction, which will impede overall productivity. Moreover, it is generally known that functional heterologous expression of p450 enzymes in microbial (e.g.) bacterial systems is oftentimes troublesome. Lastly, the use of a TAL enzyme at best only leads to diminishing of L-Phe-derived products such as CA, but does not provide for re-routing the metabolic flux from phenylalanine towards tyrosine.

It is an object of the present invention to provide a further improved method for the microbial production of hydroxylated aromatics from renewable carbon sources. In particular, it is an aim to achieve a high production level of a L-tyrosine derived product, like PHCA, PHB, PHS, and/or PHSO, with only a minimal production of unwanted (non-hydroxylated) by-products, like CA. Preferably, the production level of the desired product(s) is at least 10-fold higher than that of the by-product(s).

These goals are met by the finding that a microbial host cell is advantageously modified such that de novo synthesis of L-phenylalanine (abbreviated to L-Phe or Phe) is impeded. This approach is fundamentally different from those in the prior art and has three important implications: first, by-product formation from L-phenylalanine can be decreased or eliminated. Second, the metabolic flux of carbon is re-routed from L-phenylalanine towards L-tyrosine, leading to an enhanced production of L-tyrosine derived products (e.g. PHCA, PHS and PHSO). Third, the growth rate of the host cell and the production level of the desired product(s) can be controlled by exogenous L-phenylalanine feeding to the bacterial host.

Herewith, the invention discloses a novel methodology for decreasing by-product formation, concomitant increasing carbon flux to a central metabolite (L-tyrosine) and a manner for controlling growth and product formation in a bacterial host with a broad metabolic potential for the optimized production of various substituted aromatics.

Provided is a microbial host cell capable of producing at least one para-hydroxylated aromatic from a renewable carbon source, wherein at least one enzyme of said host cell that is involved in the degradation of said at least one hydroxylated aromatic is disabled and wherein the de novo synthesis of L-Phe in said host cell is impeded.

A host cell of the invention is capable of producing at least one para-hydroxylated aromatic from a renewable, fermentable, carbon source. To that end, the host cell comprises phenylalanine ammonia lyase (PAL) activity to allow for, among others, the conversion of L-Tyr to PHCA. The microbial host cell is for example a bacterial host cell, preferably a Gram-negative bacterium. However, other microbial cells may also be used.

The expression that the de novo L-Phe synthesis in the host cell “impeded” is meant to indicate that the host cell has no or very low endogenous capacity to synthesize L-Phe. This effect is specific for L-Phe, i.e. the capacity to synthesize L-Tyr is not or only minimally affected. A reduction or total block of microbial L-Phe synthesis can be achieved by the (genetic) modification of a host cell. Preferably, the modified host cell displays less than 10%, more preferably less than 5%, most preferably less than 1%, of the de novo L-Phe synthesis relative to the non-modified host cell. In one embodiment, the host cell is bradytrophic for L-Phe, meaning that the host cell requires exogenous L-Phe for optimal growth. In the absence of exogenous L-Phe, a bradytrophic host cell can grow yet at a highly reduced rate. In another embodiment, a host cell of the invention is auxotrophic for L-Phe, meaning that exogenous L-Phe is a prerequisite for the host cell to grow. Method to provide L-Phe bradytrophic or auxotrophic host cells are known in the art. It may involve the generation of a library of mutants using random mutagenesis, for example using UV radiation or, as exemplified herein, a chemical mutagen such as N-methyl-N′-nitro-N-nitrosoguanidine (NTG). The library containing a large population of randomly generated mutants can subsequently be screened for the requirement of exogenous L-Phe.

The term “aromatic” as used herein refers to a chemical compound having a ring structure in which some of the bonding electrons are delocalized. The at least one hydroxylated aromatic is for example selected from the group consisting of p-hydroxycinnamic acid (PHCA), p-hydroxybenzoic acid (PHB), p-hydroxystyrene (PHS) and p-hydroxystyrene oxide (PHSO).

In a preferred embodiment, a host cell according to the invention comprises an efflux pump that is capable of actively transporting said hydroxylated aromatic out of the host. A host cell comprising an efflux pump can secrete the aromatic into the culture medium such that product accumulation in the cell and, conceivably, product inhibition, is minimized. As a result, higher product yields can be achieved compared to host cell which cannot effectively secrete the synthesized hydroxylated aromatic. In addition, the use of a host cell comprising an efflux pump does not require the harvest and further processing of host cells to obtain the desired end product. Instead, the culture medium of the host cell enriched with the end product can be taken and subjected to further processing to isolate and/or purify the product. Of particular interest are host cells which display a resistant phenotype towards hydrophobic solvents, such as toluene and octanol. However, also (bacterial) host cells which are not solvent-resistant but which do comprise an efflux pump capable of exporting hydroxylated aromatics are encompassed.

Many different mechanisms have been described that contribute to solvent resistance, one of which relates to an energy-dependent efflux pump which actively keeps toxic solvents out of the interior of the cell. Solvent resistant or tolerant host cells are advantageously used in a method of the invention because the pump conferring resistance or tolerance towards organic solvents has been shown to possess a very broad specificity, taking organic compounds that by virtue of their chemico-physical characteristics accumulate into the bacterial membrane, such as aromatics, alcohols, alkanes etc., as a substrate (Kieboom et al. 1998. J. Biol. Chem. 273:85-91). Undissociated aromatic compounds will by virtue of similar chemico-physical characteristics also partition effectively to the cell membrane where they act as a substrate of such a pump.

In one embodiment of the invention, a host cell, preferably a Gram-negative bacterium, comprises a member of the proton-dependent resistance/nodulation/cell division (RND) family of efflux pumps. RND-type efflux pumps belong to the multidrug resistance (MDR) pumps. They have an extremely broad substrate specificity and protect bacterial cells from the actions of antibiotics on both sides of the cytoplasmic membrane. Members of this family have been shown to be involved in export of antibiotics, metals, and oligosaccharides involved in nodulation signaling. RND-type efflux pumps usually function as three-component assemblies spanning the outer and cytoplasmic membranes and the periplasmic space of Gram-negative bacteria. Examples of suitable RND-type efflux pumps for use in a method of the invention can be found in Tseng, T. T., Gratwick, K. S., Kollman, J., Park., D., Nies, D. H., Goffeau, A., & Saier Jr., M. H. (1999) The RND permease superfamily: an ancient, ubiquitous and diverse family that includes human disease and development proteins. J. Mol. Microbiol. Biotechnol. 1: 107-125.

In one embodiment, the host cell comprises a solvent resistance pump, preferably the solvent resistance pump srpABC of P. putida S12 (Isken et al. 1996 J. Bacteriol. 178:6056; Kieboom et al. 1998. J. Biol. Chem. 273:85-91). The srpABC pump was shown to extrude a wide variety of compounds with unrelated structures, such as aromatics, alkanes and alcohols. The deduced amino acid sequences of the proteins encoded by the srpABC genes have extensive homology with those of the RND family of efflux pumps. It is composed of three protein components that together span the inner and outer membranes of Gram-negative bacteria: an inner membrane transporter (SrpB analogues), an outer membrane channel (SrpC analogues), and a periplasmic linker protein (SrpA analogues). Dendrograms showing the phylogenetic relationship of SrpA, SrpB, and SrpC to other proteins involved in multidrug resistance are shown in Kieboom et al. 1998 J. Biol. Chem. 273:85-91. The srpABC-encoded proteins show high homology with those for the mexAB/oprM-encoded multidrug resistance pump found in Pseudomonas aeruginosa. SrpA, SrpB, and SrpC are 57.8, 64.4, and 58.5% identical to MexA, MexB, and OprM, respectively. In one embodiment of the present invention, a host cell comprises an efflux pump consisting of an inner membrane transporter, an outer membrane channel, and a periplasmic linker protein belonging to the RND-family of efflux pumps wherein the proteins show a homology of at least 50%, preferably at least 55% to the SrpA, SrpB or SrpC proteins of P. putida S12. In fact, any functional equivalent of known solvent efflux pumps that can use a hydroxylated aromatic as a substrate is suitably used.

In addition to being deficient in endogenous L-Phe synthesis, a host cell of the invention is disabled in at least one enzyme activity which is involved in the catabolism of the desired hydroxylated aromatic. This enhances accumulation of the desired product. In one embodiment, at least one enzyme in the degradation route of PHCA is disabled. As shown herein below, the gene encoding feruloyl-CoA synthase (fcs), the first enzyme involved in PHCA degradation, can be inactivated to enhance PHCA production. Likewise, depending on the hydroxylated aromatic product of interest, other catabolic enzymes can be inhibited. Preferably, at least the first enzyme involved in the degradation of the desired product is inhibited or completely blocked. In one aspect, at least one enzyme in the degradation route of PHB can be disabled, for example by inactivating or disrupting the gene encoding PHB-hydroxylase (pobA). In another embodiment, at least one enzyme in the degradation route of PHS is inactivated, for instance by gene disruption of the gene encoding styrene mono-oxygenase (smo). This leads to elimination of degradation of PHS. Subsequently, PHS production can be obtained by providing the host cell with a heterologous gene encoding PHCA decarboxylase (pdc), preferably pdc from Lactobacillus plantarum.

A further aspect of the invention relates to the use of a host cell as disclosed herein for the manufacture of substituted aromatics from fermentable feedstock. Provided is a method for the microbial production of at least one hydroxylated aromatic from a renewable carbon source, comprising providing a bacterial host cell according to the invention, culturing said host cell in the presence of exogenous L-Phe and a renewable carbon source; and allowing said host cell to produce said at least one hydroxylated aromatic.

Various carbon sources can be used to culture a host cell of the invention, provided that it can be fermented by the host cell. For example, the (renewable) carbon source is selected from the group consisting of monosaccharides, oligosaccharides, polysaccharides, polyols (like glycerol), preferably glucose and glycerol. A host cell can also be cultured on a mixture of two or more renewable, fermentable carbon sources.

In a preferred embodiment, the step of providing said host cell comprises the use of random selecting an organism which has an increased resistance against a toxic analog of an aromatic amino acid, preferably m-fluorophenylalanine (MFP) and/or m-fluorotyrosine (MFT). This procedure selects for host cells which have an increased metabolic flux towards the biosynthesis of aromatic amino acids. In combination with the L-Phe deficiency, a host cell of the invention is specifically tailored to produce para-hydroxylated aromatics without an accompanying increase in L-Phe-derived metabolites. As is exemplified below, a method of the invention allows for a very attractive ratio between the amount of desired hydroxylated product(s) synthesized and the unwanted non-hydroxylated by-product(s). For example, L-Tyr derived PHCA accumulated to a level of 860 μM whereas the non-hydroxylated, L-Phe-derived metabolite CA only reached a level of 70 μM (see Example 3). Herewith, the invention provides for a method wherein the host cell produces the at least one hydroxylated aromatic in molar excess of an L-Phe derived aromatic, in particular cinnamic acid (CA). Importantly, in a method of the invention the host cell produces said at least one hydroxylated aromatic at a sustained (i.e. non-transient) level.

In a specific aspect the invention provides a method for the manufacture of a hydroxylated aromatic comprising culturing a host cell of the invention under fed-batch fermentation conditions. In fed-batch culture, nutrients are continuously or semi-continuously added to a culture system, while effluent is removed discontinuously. It is usually used to overcome substrate inhibition or catabolite repression. Advantages of fed-batch culturing include the following. 1. High cell densities can be obtained due to extension of working time. 2. Controlled condition for the provision of substrate(s) during the fermentation. 3. Control over the production of by-products, or catabolite repression effects due to the limited provision of only those substrates solely required for product formation. 4. Allows the replacement of water lost via evaporation. 5. No additional or special pieces of equipment are required to convert form batch to fed-batch operation.

Very good results were obtained with a method of the invention comprising feeding the host cell during a first cultivation stage with exogenous renewable carbon, e.g. glucose, and L-Phe feeding until an optimal biomass is obtained, followed by feeding the host cell during a second cultivation stage with a renewable carbon source, preferably in the absence of exogenous L-Phe. Optimization of fed-batch conditions using different feed rates of L-Phe resulted in the establishment of culturing conditions with an optimal balance between growth rate, biomass yield, hydroxylated product yield and prevention of by-product formation. For the production of PHCA by a P. putida S12 host cell it was found that first cultivation stage with L-Phe at a feed rate of between 0.5-2.5 mg/L/h, for example at a feed rate of 1.5 mg/L/h.] in a mineral glucose medium resulted in a very high final concentration of PHCA while the production of CA was more than 70-fold lower. Also, the PHCA yield on L-Phe was high (30 moles/mole). This yield of hydroxylated aromatic, accompanied with a very high ratio between hydroxylated and non-hydroxylated aromatic, is clearly unsurpassed. However, depending on several factors e.g. host cell, desired product(s), carbon source and the like, other feed rates may also be used. The skilled person will be able to determine optimal feed rate for a specific situation using his routine skills.

### LEGENDS TO THE FIGURES

FIG. 1. Physical map of pTacpal. The pal gene from Rhodosporidium toruloides was cloned downstream of the tac promoter. Abbreviations: rep is required for plasmid replication; Gm^{r} is the gentamycin resistance gene; bla encodes for beta-lactamase that confers resistance to ampicillin.

FIG. 2: Transient production of PHCA (squares) and growth (triangles) in MMG in shakeflasks by P. putida S12pal (panel A) and P. putida S12C1 selected for an increased carbon flux to tyrosine (panel B). The data points are averages of triplicate experiments. Error bars indicate ±SD of the mean. CDW; cell dry weight.

FIG. 3. Sustained production of PHCA (squares) and growth (triangles) in MMG in shakeflasks by P. putida S12C2 wherein PHCA degradation is eliminated. The data points are averages of triplicate experiments. Error bars indicate ±SD of the mean. CDW; cell dry weight.

FIG. 4. Production of PHCA (squares) and growth (triangles) in MMG supplemented with 10 mg/L phenylalanine in shakeflasks by the L-Phe bradytrophic strain P. putida S12C3. The data points are averages of triplicate experiments. Error bars indicate ±SD of the mean. CDW; cell dry weight.

FIG. 5. Production of PHCA (squares), CA (diamonds) and biomass (triangles) by P. putida S12C3 during phenylalanine limited fed-batch cultivation in a mineral glucose medium.

FIG. 6. Production of PHB (circles) and biomass (triangles) by S12B1 during shakeflask incubation in MMG. OD600; optical density of the culture at 600 nm.

FIG. 7. A physical map of plasmid pTacpalpdc. Pdc from Lactobacillus plantarum DSM20174 has been amplified by PCR from the genomic DNAs with primers obtained from cloned with its own ribosomal binding site immediately downstream of rep. Abbreviations: rep is required for plasmid replication; Gm^{r} is the gentamycin resistance gene; bla encodes for beta-lactamase that confers resistance to ampicillin.

### EXPERIMENTAL SECTION

### Materials and Methods

Strains, plasmids and culture conditions. The strains and plasmids used in this study are shown in Table 1. The media that were used were Luria-Bertani broth (LB) (Sambrook et al., 1989) and a phosphate buffered mineral medium as described previously (Hartmans et al., 1989). In mineral media, 20 mM glucose was used as the sole source of carbon (MMG), unless stated otherwise. For cultivation of L-phenylalanine bradytrophs 10 mg/L L-phenylalanine was added to the medium (MMGP). Antibiotics were added as required to the media at the following concentrations: ampicillin, 100 μg/ml; gentamycin, 10 μg/ml (MMG) and 25 μg/ml (LB); tetracycline, 10 μg/ml (E. coli) and 30 μg/ml (P. putida).

TABLE 1
Bacterial strains and plasmids used in this study

| | | Reference |
|---|---|---|
| Strain or plasmid | Relevant characteristics^{a} | and/or source |
| Strains | | |
| Pseudomonas putida | P. putida S12 containing plasmid pTacpal | Nijkamp et |
| S12pal | | al., (2005)^{b} |
| P. putida S12 C1 | Derived from P. putida S12pal by NTG | This study |
| | mutagenesis and MFP selection | |
| P. putida S12 C2 | fcs knockout strain derived from P. putida S12 C1 | This study |
| P. putida S12 C3 | L-Phenylalanine bradytrophic strain derived from | This study |
| | P. putida C2 | |
| P. putida S12PHS | P. putida S12 C3 containing pTacpalpdc | This study |
| P. putida S12ΔS PHS | P. putida S12 C3 containing pTacpalpdc and smo | This study |
| | knockout | |
| P. putida S12 B1 | pobA knockout strain derived from P. putida S12 | This study |
| | C1 | |
| P. putida S12 B2 | pobA knockout strain derived from P. putida | This study |
| | S12tpl | |
| P. putida S12tpl | Cured strain of P. putida S12tpl3 | Wierckx et al. |
| | | (2005) |
| Escherichia coli DH5α | supE44 ΔlacU169 (φ80 lacZΔM15) hsdRl7 recA1 | Sambrook et |
| | endA1 gyrA96 thi-1 relA1 | al. (1989) |
| Plasmid | | |
| pGEM-T Easy | Ap^{r}, used for cloning PCR fragments | Promega |
| pTO1 | Tet^{r}, used for amplification of tetA | Kieboom and |
| | | De Bont |
| | | (2001) |
| pTacpal | Ap^{r} Gm^{r}, expression vector containing the pal | Nijkamp et al. |
| | gene under control of the tac promoter | (2005)^{b} |
| pTacpalpdc | Ap^{r} Gm^{r}, expression vector containing the pal | |
| | gene under control of the tac promoter and pdc | |
| | with RBS behind rep | |
| pTnModKMO | Km^{r}, used for amplification of Km | Dennis and |
| | | Zylstra (1998) |
| pJQ200SK | Suicide vector, P15A ori sacB RP4 Gm^{r} | Quandt and |
| | pBluescriptSK MCS | Hynes (1993) |
| pJQfcs::tet | pJQ200SK containing the tetA interrupted fcs | This study |
| | gene | |
| pJQpobA::tet | pJQ200SK containing the tetA interrupted pobA | This study |
| | gene | |

^{a}Ap^{r}, Gm^{r} and Tet^{r}, ampicillin, gentamicin and tetracycline resistance respectively.
^{b}plasmid pTacpal has been erroneously exchanged with pJWpalTn in the study of Nijkamp et al. (2005).

Shakeflask experiments were performed in 250 ml erlenmeyer flasks containing 50 ml of MMG in a horizontal shaking incubator at 30° C. Cultures were inoculated to a starting OD₆₀₀ of 0.2 with cells from an overnight culture. Fed-batch experiments were performed in 2 L fermentors (New Brunswick Scientific) using a BioFlo110 controller. Initial batch fermentation was started from a 50 ml inoculum of an overnight culture in MMG+100 mg/L L-phenylalanine. For the batch phase an adapted mineral medium was used with the following composition (per litre): 36 g glucose, 4 g (NH₄)₂SO₄, 3.88 g K₂HPO₄, 1.63 g NaH₂PO₄.H₂O and 20 ml trace element solution. The trace element solution had the following composition (per litre): 10 g MgCl₂.6H₂O, 1 g EDTA, 0.2 g ZnSO₄.7H₂O, 0.1 g CaCl₂.2H₂O, 0.5 g FeSO₄.7H₂O, 0.02 g Na₂MoO₄.2H₂O, 0.02 g CuSO₄.5H₂O, 0.04 g CoCl₂.6H₂O, 0.1 g MnCl₂.4H₂O. Growth was controlled by addition of L-phenylalanine. After depletion of the initial glucose, the L-phenylalanine feed was stopped and a glucose feed was started. The stirring speed was set to 200 rpm and air was supplied at 1 L/min. Dissolved oxygen tension was kept on 15% air saturation by automatic adjustment of the stirring speed and mixing with pure oxygen. Medium samples (5 ml) were taken during the fermentation to determine cell dry weight (CDW), glucose, ammonium, PHCA and CA concentration. CO₂ and O₂ concentrations in the offgas were measured using an Innova 1313 Fermentation Monitor. The pH was maintained at 7.0 with 4 N KOH and 4 N HCl.

Analytical methods. Cell densities were measured at 600 nm (pathway length 1 cm) with a Biowave Cell Density Meter (WPA Ltd). CDW concentrations were calculated from OD600 values using the formula CDW (g/L)=OD600×0.465. CA, PHCA, PHB and PHS concentrations were analyzed by HPLC (Agilent 1100 system) using a Zorbax 3.5 μm SB-C18 column (4.6×50 mm) with acetonitril: NaH₂PO₄-buffer (50 mM, pH 2, 1% acetonitril) (25:75 for CA, PHCA, PHS and 17:83 for PHA) as an eluent. Glucose concentrations were analyzed by HPLC (Waters) using an Aminex HDP-87N column with 0.01 M Na₂HPO4 as an eluent. Gluconic acid and 2-ketogluconic acid concentrations were analyzed by HPLC (Waters) using an Aminex HDP-87H column with 0.008 N H₂SO₄ as an eluent. NH₄_{⁺} concentrations were determined by cation-exchange chromatography (Dionex).

DNA techniques. The suicide vector pJQ200SK (Quandt and Hynes, 1993) was used to construct a gene replacement vector for the fcs gene as described below. Primers JW1-JW4 (See Table 2 for primer characteristics), based on the known DNA sequence of fcs from P. putida KT2440 (Weinel et al., 2002), were used to amplify the first 825 bp (fcs1) and the last 870 bp (fcs2) of the fcs gene. The tetracycline resistance gene (tetA) from vector pTO1 (Kieboom and De Bont, 2001) was amplified using primers JW5 and JW6 (Table 2). The three PCR products were ligated in pGEM-T Easy (Promega). pJQ200SK was digested with NotI and BamHI and fcs1 and fcs2 were cut from pGEM-T Easy with NotI/XbaI and BamHI/XbaI respectively. The three DNA fragments were then ligated to yield pJQfcs. pJQfcs was linearized with XbaI and treated with bacterial alkaline phospatase (BAP). TetA was cut from pGEM-T Easy using XbaI and ligated into the linearized pJQfcs vector to yield pJQfcs::tet. This construct was electroporated into P. putida S12 C1 and cells were plated on LB-agar plates containing tetracycline. Colonies that were Tet⁺ and Gm⁻ were selected and replacement of the fcs gene by the tetA disrupted fcs copy was confirmed by growth on PHCA as the sole source of carbon and by production of PHCA after introducing of pTacpal.

pobA knockouts were obtained essentially as described for fcs with following modification: Primers used for the PCR amplification are shown in table 2. The homologous DNA fragments of pobA, 528 bp (pobA1) and 610 bp (pobA2), were digested with the enzymes NotI/XbaI and XbaI/XhoI. Tet⁺ and Gm⁻ colonies were tested for growth on PHB as sole carbon source and for the production of PHB after introducing of pTacpal.

S12 strains with a disrupted copy of smo were obtained essentially as described as above with following modifications: Primers used for the amplification are shown in table 2. The first 590 bp and the last 585 bp DNA fragments of smo (designated as smo1 and smo2) were amplified by PCR and digested with NotI/XbaI and XbaI/BamHI, respectively. A kanamycin resistance gene (Km^{r}) was used for disruption of smo1/2. The gene pdc, was amplified from the genomic DNA of Lactobacillus plantarum DSM20174 by PCR using primers MW7 and MW8 and cloned just downstream of the rep gene in pTacpal.

TABLE 2
Primers used in this study.

| | | |
|---|---|---|
| Primer | Sequence (3′ → 5′)^{a} | Characteristics |

| | | |
|---|---|---|
| JW1 | gcgcggccgcatgcaacctgtcgagccactggcg | Start of fcs, forward primer, NotI |
| JW2 | gcgtctagactcgcgcagattgcgcaaggtctc | Pos. 800-825 bp in fcs, reverse primer, XbaI |
| | gg | |
| JW3 | gcgtctagactacgcgaggtgttctttgcccgca | Pos. 901-927 bp in fcs, forward primer, XbaI |
| | tc | |
| JW4 | gcgggatcctcaaggccgcaccttggcgtgcaa | End of fcs, reverse primer, BamHI |
| | tgc | |
| JW5 | gcgtctagactcaggtcgaggtggcccgg | Start of tetA from pTO1 (Kieboom and De Bont |
| | | 2001), forward primer, XbaI |
| JW6 | gcgtctagagaattctcatgtttgacagcttatc | End of tetA from pTO1 (Kieboom and De Bont |
| | | 2001), reverse primer, XbaI |
| JW7 | gcgcggccgcatgaaaactcaggttgcaattat | Start of pobA, forward primer, NotI |
| | tgg | |
| JW8 | gcgtctagactgtttcagcacgccctccggg | Pos. 528-507 bp in pobA, reverse primer, XbaI |
| JW9 | gcgtctagacgccagtcaatcacgagttgatc | Pos. 578-600 bp in pobA, forward primer, XbaI |
| JW10 | gggctcgagtcaggcaacttcctcgaacggc | End of pobA, reverse primer, XhoI |
| MW1 | gcggcggccgcatgaaaaagcgtatcggtatt | Start of smo, forward primer, NotI |
| | gttg | |
| MW2 | gcgtctagatcaatcagctcgccatgccctg | Pos. 569-590 bp in smo, reverse primer, XbaI |
| MW3 | gcgtctagagaagttctcgcccacaccaag | Pos. 661-681 bp in smo, forward primer, XbaI |
| MW4 | gcgggatcctcaggccgcgatagtcggtgc | End of smo, reverse primer. BamHI |
| MW5 | gcgtctagaatgagccatattcaacgggaaac | Start of Km from TnModKMO (Dennis and |
| | g | Zystra 1998), forward primer, XbaI |
| MW6 | gcgtctagattagaaaaactcatcgagcatca | End of Km from TnModKMO (Dennis and |
| | aatg | Zystra 1998), reverse primer, XbaI |
| MW7 | gcggcggccgcgacataaggaaggtaattcta | Leader sequence + ribosomal bindingsite + start |
| | atgac | pdc Lactobacillus plantarum DSM20174, Not I, |
| | | forward |
| MW8 | gcggctagcttacttatttaaacgatggtagttt | End pdc Lactobacillus plantarum DSM20174, |
| | tg | NheI, reverse |

^{a}Restriction sites are underlined.

### Examples

### Example 1

### Isolation and Characterisation of a PHCA Overproducing Mutant Strain of Pseudomonas putida S12

Pseudomonas putida S12 is able to produce CA and minute amounts of PHCA from glucose via L-phenylalanine and L-tyrosine, respectively, upon introduction and expression of the pal gene (P. putida S12pal) coding for L-phenylalanine ammonia lyase from Rhodosporidium toruloides (Nijkamp et al., 2005, WO2005/103273). It was shown previously that CA production in such a strain was greatly enhanced after a combination of NTG treatment and selection on MFP, which selects for mutants with an enhanced metabolic flux towards L-phenylalanine (Nijkamp et al., 2005, WO2005/103273).

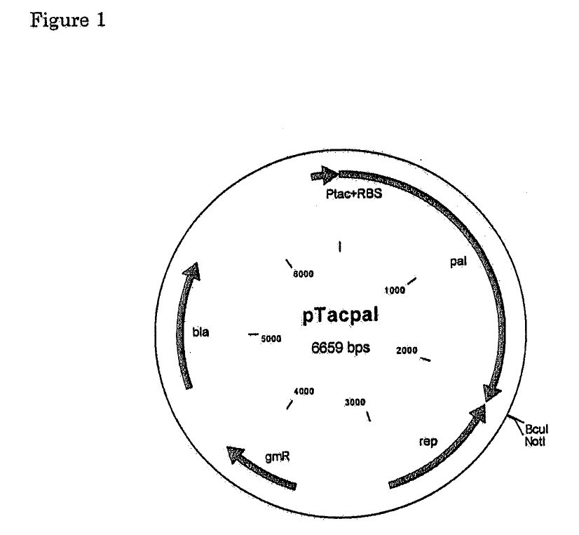

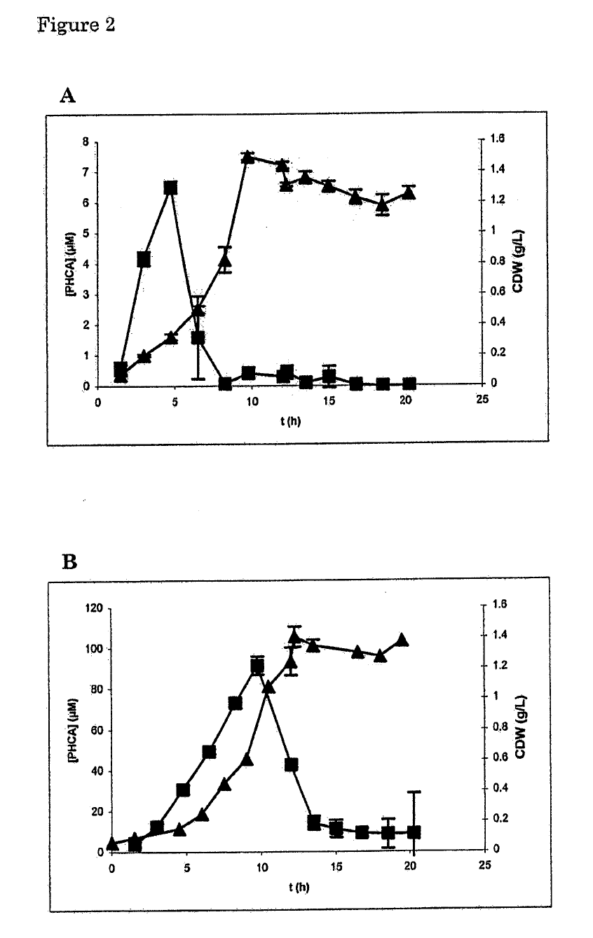

Given the common biosynthetic pathway of L-tyrosine and L-phenylalanine, it was initially anticipated that this procedure would also yield mutants with an increased carbon flux to L-tyrosine and concomitant PHCA production. A library of 11000 pal expressing (via plasmid pTacpal: FIG. 1) MFP resistant mutants of P. putida S12 was screened for PHCA production. Hereto, the mutants were cultivated for 8 hours in mineral glucose medium (MMG) in microtiter plates. The presence of PHCA in de cultures supernatants was determined by measuring the absorbance at 310 nm. Positive mutants were subsequently cultivated in shakeflasks to confirm increased PHCA production by HPLC. Mutant S12C1 was found to accumulate the highest levels of PHCA: a maximum PHCA concentration of 90 μM was reached after 10 hours of growth in MMG (FIG. 2B), which is a 14-fold increase in production when compared to its parent strain P. putida S12pal (FIG. 2A). However, after 24 hours almost all PHCA was degraded. Thus, the increase in PHCA production was only transient whereas a stable, sustained production is of course preferred. P. putida S12C1 grew poorly on PHCA as sole carbon source compared to P. putida S12 wildtype (results not shown). Growth on p-hydroxybenzaldehyde and PHB, intermediates in the degradation pathway of PHCA in P. putida (Jiminez et al., 2002), as sole sources of carbon, was comparable to wild type S12 (results not shown). This result indicated that accumulation of PHCA in S12C1 finds its origin in the hampered conversion of the compound in the first step(s) of the degradation pathway.

### Example 2

### Construction and Characterization of a Host Cell Capable of Stably Producing High Levels of PHCA

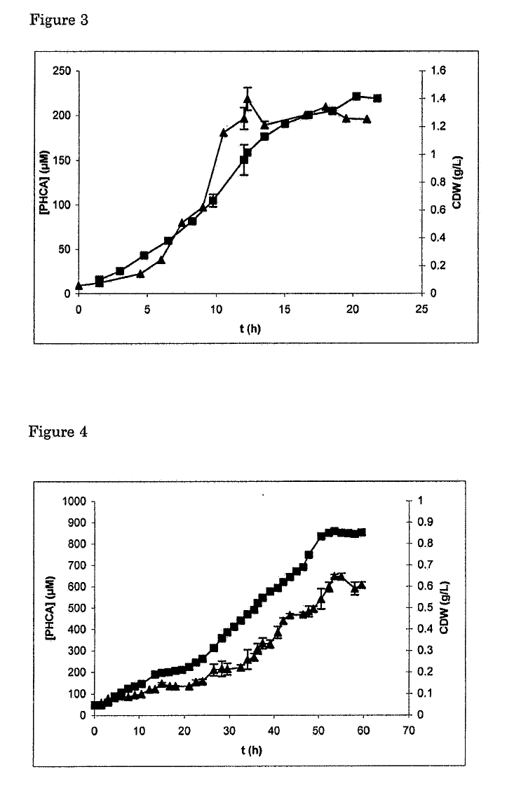

To overcome the problem of transient PHCA production caused by PHCA degradation as described in Example 1, the gene feruloyl-CoA synthase (fcs) encoding the first conversion in the PHCA catabolic pathway in P. putida (Jiminez et al., 2002) was inactivated in strain S12C1. Plasmid pJQ200SK (Quandt and Hynes, 1993) was used as a delivery system for gene replacement by homologous recombination of the wildtype fcs allele by a tetA-cassette disrupted copy. P. putida S12C1, cured from pTacpal, was electrotransformed with this construct and the resulting Tet^{r} clones were tested for the ability to grow on PHCA and for Gm^{r}, the marker for pJQ200SK. Several Gm^{s} clones unable to utilize PHCA were isolated. The successful replacement of fcs with the inactivated copy (fcs::tet) was confirmed by PCR analysis (not shown). One mutant was electrotransformed with pTacpal and the resulting transformant was designated P. putida S12C2. This transformant was found to stably accumulate 224 μM PHCA during shakeflask cultivation in MMG. However, also 350 μM of CA was formed (not shown), indicating a considerable flux of carbon towards L-phenylalanine in S12C2 (FIG. 3).

### Example 3

### Generation and Screening of a Library of L-phenylalanine Bradytrophic Mutants of P. putida S12C2 for Increased PHCA-Production and Decreased Production of the By-Product CA

In order to prevent formation of the by-product CA in S12C2 and to increase the metabolic carbon flux from glucose towards L-tyrosine, a strategy was chosen to prevent de novo synthesis of L-phenylalanine in this strain. S12C2 was cured from pTacpal and subsequently treated with NTG in order to obtain a large population of randomly generated mutants. The mutants were plated on MMG medium agar supplemented with 1 mg/L L-phenylalanine. Small to pinpoint colonies arose at a frequency of approximately 10% compared to colonies of normal size. Three thousand pinpoint colony forming mutants were tested for their ability to grow in MMG supplemented with 100 mg/L L-phenylalanine or with 100 mg/L L-tyrosine. Four mutants able to grow in the medium with L-phenylalanine only (L-phenylalanine bradytrophic strains or phe-strains) were selected. After reintroduction of plasmid pTacpal into the phe-strains, PHCA production in MMG supplemented with 10 mg/L phenylalanine (MMGP) was monitored. One strain, designated P. putida S12C3, showed a dramatically improved PHCA production: 860 μM of PHCA was produced in MMGP during incubation in shakeflasks (FIG. 4). This was a 4-fold increase in production compared to P. putida S12C2. Moreover, in this strain the final CA concentration was 70 μM (not shown), a 5-fold decrease compared to S12C1 and S12C2.

### Example 4

### Optimization of Fed-Batch Conditions for Hydroxylated Aromatic Production

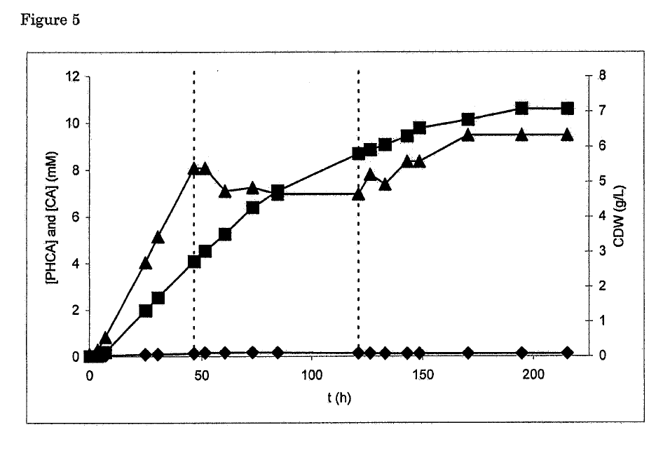

The production of PHCA by P. putida S12C3 with glucose as the sole source of carbon was studied in fed-batch fermentations. Since P. putida S12C3 is phe-, L-phenylalanine limiting conditions were applied. In S12C3 excess phenylalanine could also be transformed to CA by L-phenylalanine ammonia lyase. In order to find the optimal balance between growth rate, biomass yield, PHCA yield and prevention of CA formation, fed-batch experiments using different L-phenylalanine feed rates were performed (not shown). An optimal L-phenylalanine feed rate of 1.5 mg/h/L was found. During the first process stage (FIG. 5, I) L-phenylalanine was fed to the culture to allow for biomass formation and production of PHCA. In the next stage (FIG. 5, II) the L-phenylalanine feed was stopped and a glucose feed was started with a rate of 1 g/h. We observed production of PHCA under no-growth conditions in this stage. Both the PHCA and biomass yield on glucose reached their maximum at the end of stage II. In stage III of the fed-batch process (FIG. 5, III) we observed an increase in biomass concentration.

This resulted in a final concentration of 10.6 mM of PHCA (FIG. 5) with a maximum Y_{p/s} of 3.8% (Cmol %). Furthermore, only 150 μM of CA was formed, resulting in a PHCA to CA ratio of 85 moles/mole. Finally, the PHCA yield on L-phenylalanine was 30 moles/mole and the biomass yield on L-phenylalanine was 75 g/g. Table 3 summarizes the PHCA production by P. putida S12 strains obtained using either shakeflask or fed-batch cultivation.

TABLE 3
Overview of the results obtained in shakeflask and fed-batch
cultivated p-coumaric acid (PHCA) producing P. putida S12 strains.

| | | | | | q_{p, max, PHCA} |
|---|---|---|---|---|---|
| | | | | | (μmol/ |
| | [PHCA]ₘₐₓ | [CA]ₘₐₓ | Y_{p/s, PHCA} | Y_{p/x, PHCA} | min/g |
| Strain | (μM) | (μM) | (Cmol %)^{a} | (g/g)^{b} | CDW)^{c} |
| S12pal | 7 | 72 | 0.05 | 8 · 10⁻⁴ | 0.3 |
| S12pal C1 | 91 | 354 | 0.7 | 0.01 | 1.4 |
| S12pal C2 | 224 | 314 | 1.7 | 0.03 | 0.5 |
| S12pal C3 | 860 | 70 | 6.5 | 0.23 | 1.4 |
| S12pal C3 | 10600 | 150 | 3.3 | 0.30 | 0.4 |
| fed-batch | | | | | |

^{a}yield in Cmol PHCA per Cmol glucose used × 100%.
^{b}yield in g PHCA per g cell dry weight.
^{c}maximum specific PHCA production rate calculated by the formula qₚ= rₚ/Mₓ (33).
rₚ is the PHCA production rate (μmol/L/min) and Mₓ is the biomass concentration (g/L).

### Example 5

### Construction and Characterization of PHB Hydroxylase Deficient Derivatives of P. putida S12C1 and P. putida S12tpl3

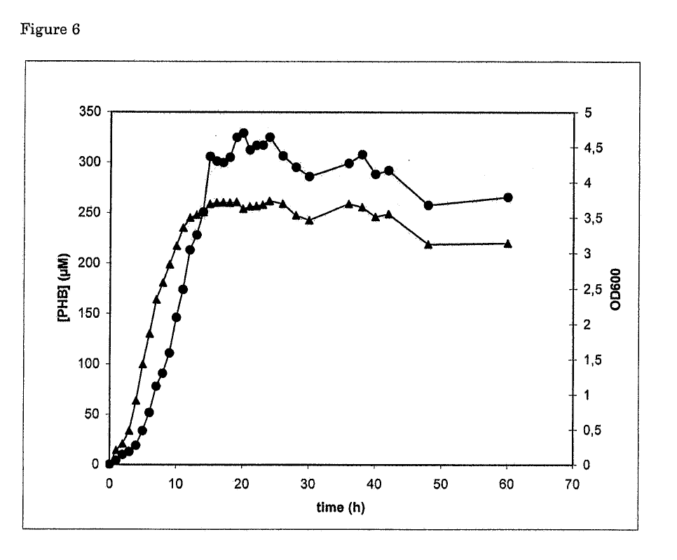

To completely prevent PHB degradation in strain S12C1 (Example 2) and strain S12tpl3, that was previously optimized for the enhanced metabolic flux towards L-tyrosine through random mutagenesis and screening approaches followed by selection on MFP and MFT (Wierckx et al., 2005), the gene PHB-hydroxylase (pobA) encoding the first conversion in the PHB catabolic pathway in P. putida (Jiminez et al., 2002) was inactivated after curation of both strains from their plasmids. This was achieved essentially via the gene replacement methodology described in example 2, but tailored for pobA inactivation. After introduction of pTacpal in obtained pobA deficient strains, derivates were obtained, designated S12B1 (derived from S12C1) and S12B2 (derived from S12tpl3), that accumulated PHB during shakeflask incubation in MMG (FIG. 6). However, also a considerable amount (appr. 400 μM) of CA was produced (not shown), indicating a significant flux of carbon towards phenylalanine in strain B1. Therefore, L-Phe brady/auxotrophic variant host cells are prepared as described in Example 3. Fed-batch experiments are performed to optimize culturing conditions for PHB production with a minimal amount of CA production.

### Example 6

### Construction and Characterization of a PHS Producing Derivative from S12C3

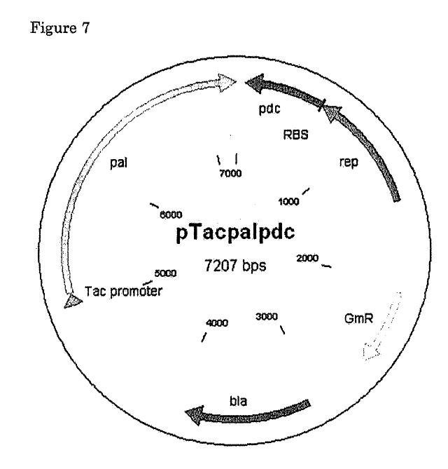

Strain S12C3 (Example 3), cured from plasmid pTacpal, was electrotransformed with plasmid pTacpalpdc (FIG. 7) for the heterologous expression of both the pal gene and the pdc gene from Lactobacillus plantarum. The pdc gene encodes for PHCA decarboxylase, which converts PHCA into PHS (Cavin et al., 1997). Thus obtained strain S12 PHS was able to produce up to 0.6 mM PHS from glucose in MMG supplemented with 100 mg/ml L-phenylpyruvate or L-phenylalanine during shakeflask incubation. Under these batch conditions approximately 0.3 mM of CA and PHCA accumulated (not shown). Fed-batch experiments can be used (see example 4) to adjust L-phenylalanine feed such that the formation of CA is further minimized and PHCA is completely converted to PHS.

### REFERENCES

Anastas, P. T., and M. M. Kirchhoff. 2002. Origins, current status, and future challenges of green chemistry. Acc Chem Res 35:686-694.
Barker, J. L., and J. W. Frost. 2001. Microbial synthesis of p-hydroxybenzoic acid from glucose. Biotechnol Bioeng 76:376-390.
Ben-Bassat, A., and D. J. Lowe. Nov. 18, 2004. Method for producing para-hydroxystyrene and other multifunctional aromatic compounds using two-phase extractive fermentation. USA patent US 2004/0229326.
Cavin, J-F., Barthelmebs, L., Guzzo, J., Van Beeumen, J., Samyn, B., Travers, J-F., and Divies, C. 1997. Purification and characterization of an inducible p-coumaric acid decarboxylase from Lactobacillus plantarum. FEMS Microbiol. Lett. 147: 291-295.
Gatenby, A. A., S. Sariaslani, X.-S. Tang, W. W. Qi, and T. Vanelli. Apr. 9, 2002 2002. Bioproduction of para-hydroxycinnamic acid. U.S. Pat. No. 6,368,837.
Hahlbrock, K., and D. Scheel. 1989. Physiology and molecular biology of the phenolpropanoid metabolism. Annu Rev Plant Physiol Mol Biol 40:347-369.
Hanson, K. R., and E. A. Havir. 1978. An introduction to the enzymology of phenylpropanoid biosynthesis. Recent Adv Phytochem 12:91-137.
Hartmans, S., J. Smits, M. van der Werf, F. Volkering, and J. A. M. de Bont. 1989. Metabolism of styrene oxide and 2-phenylethanol in the styrene-degrading Xanthobacter strain 124X. Appl Environ Microbiol 55:2850-2855.
Hodgins, D. S. 1971. Yeast phenylalanine ammonia-lyase; Purification, properties, and the identification of catalytically essential dehydroalanine. Journal of Biological Chemistry 246:2977-2985.
Isken, S., and J. A. de Bont. 1996. Active efflux of toluene in a solvent-resistant bacterium. J Bacteriol 178:6056-6058.
Jimenez, J. I., B. Minambres, J. L. Garcia, and E. Diaz. 2002. Genomic analysis of the aromatic catabolic pathways from Pseudomonas putida KT2440. Environ Microbiol 4:824-841.
Kaneko, T., M. Matsusaki, T. Hang Thi, and M. Akashi. 2004. Thermotrophic liquid-crystalline polymer derived from natural cinnamoyl biomonomers. Macromol Rapid Commun 25:673-677.
Kieboom, J., and J. A. M. de Bont. 2001. Identification and molecular characterization of an efflux system involved in Pseudomonas putida S12 multidrug resistance. Microbiology 147:43-51.
Kieboom, J., J. J. Dennis, J. A. de Bont, and G. J. Zylstra. 1998. Identification and molecular characterization of an efflux pump involved in Pseudomonas putida S12 solvent tolerance. J Biol Chem 273:85-91.
Masai, E., K. Harada, X. Peng, H. Kitayama, Y. Katayama, and M. Fukuda. 2002. Cloning and characterization of the ferulic acid catabolic genes of Sphingomonas paucimobilis SYK-6. Appl Environ Microbiol 68:4416-4424.
Matsusaki, M., A. Kishida, N. Stainton, C. W. G. Ansell, and M. Akashi. 2001. Synthesis and characterization of novel biodegradable polymers composed of hydroxycinnamic acid and D,L-lactic acid. J Appl Polymer Sci 82:2357-2364.
Matsusaki, M., T. Hang Thi, T. Kaneko, and M. Akashi. 2005. Enhanced effects of lithocholic acid incorporation into liquid-crystalline biopolymer poly(coumaric acid) on structural ordering and cell adhesion. Biomaterials 26:6263-70.
Maury, J., M. A. Asadollahi, K. Moller, A. Clark, and J. Nielsen. 2005. Microbial isoprenoid production: an example of green chemistry through metabolic engineering. Adv Biochem Eng Biotechnol 100:19-51.
Miyahisa, I., N. Funa, Y. Ohnishi, S. Martens, T. Moriguchi, and S. Horinouchi. 2005. Combinatorial biosynthesis of flavones and flavonols in Escherichia coli. Appl Microbiol Biotechnol:1-6.
Nelson, K. E., C. Weinel, I. T. Paulsen, R. J. Dodson, H. Hilbert, V. A. Martins dos Santos, D. E. Fouts, S. R. Gill, M. Pop, M. Holmes, L. Brinkac, M. Beanan, R. T. DeBoy, S. Daugherty, J. Kolonay, R. Madupu, W. Nelson, O. White, J. Peterson, H. Khouri, I. Hance, P. Chris Lee, E. Holtzapple, D. Scanlan, K. Tran, A. Moazzez, T. Utterback, M. Rizzo, K. Lee, D. Kosack, D. Moestl, H. Wedler, J. Lauber, D. Stjepandic, J. Hoheisel, M. Straetz, S. Heim, C. Kiewitz, J. A. Eisen, K. N. Timmis, A. Dusterhoft, B. Tummler, and C. M. Fraser. 2002. Complete genome sequence and comparative analysis of the metabolically versatile Pseudomonas putida KT2440. Environ Microbiol 4:799-808.
Nijkamp, K., N. van Luijk, J. A. de Bont, and J. Wery. 2005. The solvent-tolerant Pseudomonas putida S12 as host for the production of cinnamic acid from glucose. Appl Microbiol Biotechnol 69:170-177.
Qi, W. W., F. S. Sariaslani, and X. S. Tang. 14 Nov. 2002 2003. Methods for the production of L-tyrosine, cinnamic acid and para-hydroxycinnamic acid. US patent WO 02/090523 A2.
Quandt, J., and M. F. Hynes. 1993. Versatile suicide vectors which allow direct selection for gene replacement in gram-negative bacteria. Gene 127:15-21.
Ramos-Gonzalez, M. I., Ben-Bassat, A., Campos, M. J., and Ramos, J. L. 2003. Genetic engineering of a highly solvent-tolerant Pseudomonas putida strain for biotransformation of toluene to p-hydroxybenzoate. Appl. Environ. Microbiol. 69:5120-5127.
Rojas, A., Duque, E., Schmid, A., Hurtado, A., Ramos, J-L. and Segura, A. 2004. Biotransformation in double-phase systems: physiological responses of Pseudomonas putida DOT-T1E to a double phase made of aliphatic alcohols and biosynthesis of substituted catechols. Appl. Environ. Microbiol. 70:3637-43.
Rojas, A., E. Duque, G. Mosqueda, G. Golden, A. Hurtado, J. L. Ramos, and A. Segura. 2001. Three efflux pumps are required to provide efficient tolerance to toluene in Pseudomonas putida DOT-T1E. J Bacteriol 183:3967-3973.
Sambrook, J., T. Maniatis, and E. F. Fritsch. 1982. Molecular Cloning. A Laboratory Manual. Cold Spring Harbor, N.Y., Cold Spring Harbor Laboratory.
Schmid, A., J. S. Dordick, B. Hauer, A. Kiener, M. Wubbolts, and B. Witholt. 2001. Industrial biocatalysis today and tomorrow. Nature 409:258-268.
Tang, X. S., T. Vannelli, W. W. Qi, F. S. Sariaslani, and A. A. Gatenby. Feb. 18, 2003. Polynucleotide encoding a mutant Rhodotorula glutinis L-tyrosine ammonia lyase polypeptide. U.S. Pat. No. 6,521,748.
Wackett, L. P., 2003, Pseudomonas putida-a versatile biocatalyst. Nat. Biotechnol., 21:136-8.
Weinel, C., K. E. Nelson, and B. Tummler. 2002. Global features of the Pseudomonas putida KT2440 genome sequence. Environ Microbiol 4:809-818.
Wery, J. and De Bont, J. A. M. 2004. Solvent-tolerance of Pseudomonads: A New Degree of Freedom in Biocatalysis, Chapter 20. p 609-634. In: Ramos, J. L. (Ed.) Pseudomonas, Vol. 3. Kluwer Academic/Plenum Publishers, New York.
Wery, J., D. I. Mendes da Silva, and J. A. M. de Bont. 2000. A genetically modified solvent-tolerant bacterium for optimized production of a toxic fine chemical. Appl. Microbiol. Biotechnol. 54:180-5.
Wierckx, N. J., H. Ballerstedt, J. A. de Bont, and J. Wery. 2005. Engineering of solvent-tolerant Pseudomonas putida S12 for bioproduction of phenol from glucose. Appl Environ Microbiol 71:8221-7.
Zaks, A. 2001. Industrial biocatalysis. Curr Opin Chem Biol 5:130-136.

1. A microbial host cell comprising phenylalanine ammonia lyase (PAL) activity capable of producing at least one para-hydroxylated aromatic from a renewable carbon source, wherein at least one enzyme of said host cell that is involved in the degradation of said at least one hydroxylated aromatic is disabled and wherein the de novo synthesis of L-phenylalanine (L-Phe) in said host cell is impeded.

2. Host cell according to claim 1, wherein said host cell is a L-Phe bradytrophic or auxotrophic (phe-) mutant host cell.

3. Host cell according to claim 1, wherein said host cell comprises an efflux pump for said hydroxylated aromatic.

4. Host cell according to claim 1, wherein said efflux pump is a member of the proton-dependent resistance/nodulation/cell division (RND) family of efflux pumps, preferably a solvent resistant pump, more preferably the solvent resistant pump srpABC of P. putida strain S12.

5. Host cell according to claim 1, wherein at least one enzyme in the degradation route of PHCA is disabled, preferably wherein the gene encoding feruloyl-CoA synthase is inactivated.

6. Host cell according to claim 1, wherein at least one enzyme in the degradation route of PHB is disabled, preferably wherein the gene encoding PHB-hydroxylase (pobA) is inactivated.

7. Host cell according to claim 1, wherein at least one enzyme in the degradation route of PHS is inactivated, preferably wherein the gene encoding styrene mono-oxygenase (smo) is inactivated.

8. Host cell according to claim 7, wherein said host cell expresses a heterologous gene encoding PHCA decarboxylase (pdc), preferably pdc from Lactobacillus plantarum.

9. A method for the microbial production of at least one hydroxylated aromatic from a renewable carbon source, comprising the steps of:
providing a bacterial host cell according to claim 1,
culturing said host cell in the presence of exogenous L-Phe and a renewable carbon source; and
allowing said host cell to produce said at least one hydroxylated aromatic.

10. Method according to claim 9, wherein providing said host cell comprises the use of random selecting an organism which has an increased resistance against a toxic analog of an aromatic amino acid, preferably m-fluorophenylalanine (MFP) and/or m-fluorotyrosine (MFT).

11. Method according to claim 9, wherein said at least one hydroxylated aromatic is selected from the group consisting of p-hydroxycinnamic acid (PHCA), p-hydroxybenzoic acid (PHB), p-hydroxystyrene (PHS) and p-hydroxystyrene oxide (PHSO).

12. Method according to claim 9, wherein said renewable carbon source is selected from the group consisting of monosaccharides, oligosaccharides, polysaccharides, carbon-containing amines, polyols like glycerol, preferably glucose or glycerol.

13. Method according to claim 9, wherein said host cell produces said at least one hydroxylated aromatic at a sustained level.

14. Method according to claim 9, comprising culturing said host cell under fed-batch conditions, preferably under L-Phe limited fed-batch conditions.

15. Method according to claim 9, comprising feeding the host cell during a first cultivation stage with an exogenous renewable carbon source and L-Phe until an optimal biomass is obtained, followed by feeding the host cell during a second cultivation stage with a renewable carbon source, preferably in the absence of exogenous L-Phe.

16. Method according to claim 15, comprising feeding the host cell during said first cultivation stage with L-Phe at a feed rate of between about 0.5 and about 2.5 mg/L/h.

17. Host cell according to claim 2, wherein:
said host cell comprises an efflux pump for said hydroxylated aromatic;
wherein said efflux pump is a member of the proton-dependent resistance/nodulation/cell division (RND) family of efflux pumps, preferably a solvent resistant pump, more preferably the solvent resistant pump srpABC of P. putida strain S12;
wherein at least one enzyme in the degradation route of PHCA is disabled, preferably wherein the gene encoding feruloyl-CoA synthase is inactivated;
wherein at least one enzyme in the degradation route of PHB is disabled, preferably wherein the gene encoding PHB-hydroxylase (pobA) is inactivated;
wherein at least one enzyme in the degradation route of PHS is inactivated, preferably wherein the gene encoding styrene mono-oxygenase (smo) is inactivated; and
wherein said host cell expresses a heterologous gene encoding PHCA decarboxylase (pdc), preferably pdc from Lactobacillus plantarum.

18. A method for the microbial production of at least one hydroxylated aromatic from a renewable carbon source, comprising the steps of:
providing a bacterial host cell according to claim 17,
culturing said host cell in the presence of exogenous L-Phe and a renewable carbon source; and
allowing said host cell to produce said at least one hydroxylated aromatic.

19. Method according to claim 18, wherein:
said at least one hydroxylated aromatic is selected from the group consisting of p-hydroxycinnamic acid (PHCA), p-hydroxybenzoic acid (PHB), p-hydroxystyrene (PHS) and p-hydroxystyrene oxide (PHSO);
said renewable carbon source is selected from the group consisting of monosaccharides, oligosaccharides, polysaccharides, carbon-containing amines, polyols like glycerol, preferably glucose or glycerol;
said host cell produces said at least one hydroxylated aromatic at a sustained level;
the step is provided of culturing said host cell under fed-batch conditions, preferably under L-Phe limited fed-batch conditions;
the step is provided of feeding the host cell during a first cultivation stage with an exogenous renewable carbon source and L-Phe until an optimal biomass is obtained, followed by feeding the host cell during a second cultivation stage with a renewable carbon source, preferably in the absence of exogenous L-Phe; and
the step is provided of feeding the host cell during said first cultivation stage with L-Phe at a feed rate of between about 0.5 and about 2.5 mg/L/h.

20. Method according to claim 10, wherein:
said at least one hydroxylated aromatic is selected from the group consisting of p-hydroxycinnamic acid (PHCA), p-hydroxybenzoic acid (PHB), p-hydroxystyrene (PHS) and p-hydroxystyrene oxide (PHSO);
said renewable carbon source is selected from the group consisting of monosaccharides, oligosaccharides, polysaccharides, carbon-containing amines, polyols like glycerol, preferably glucose or glycerol;
said host cell produces said at least one hydroxylated aromatic at a sustained level;
the step is provided of culturing said host cell under fed-batch conditions, preferably under L-Phe limited fed-batch conditions;
the step is provided of feeding the host cell during a first cultivation stage with an exogenous renewable carbon source and L-Phe until an optimal biomass is obtained, followed by feeding the host cell during a second cultivation stage with a renewable carbon source, preferably in the absence of exogenous L-Phe; and
the step is provided of feeding the host cell during said first cultivation stage with L-Phe at a feed rate of between about 0.5 and about 2.5 mg/L/h.
